SEQUENCE LISTING (4 sequences)

SEQ ID NO: 1 - DNA, 1706 bp, Homo sapiens
gaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgcaaaaaaaaaaaaaa

SEQ ID NO: 2 - protein, 523 aa, Homo sapiens
EEHILKLCANLDAFTIKTVKENLDQLKNQIQEPCKKFAIADATREDPFKEKLLEIMTRIQTYCQMSPMSDFGTQPYEQWAIQMEKKAAKEGNRKERVCAEHLRKYNEALQINDTIRMIDAYTHLETFYNEEKDKKFAVIEDDSDEGGDDEYCDGDEDEDDLKKPLKLDETDRFLMTLFFENNKMLKRLAENPEYENEKLTKLRNTIMEQYTRTEESARGIIFTKTRQSAYALSQWITENEKFAEVGVKAHHLIGAGHSSEFKPMTQNEQKEVISKFRTGKINLLIATTVAEEGLDIKECNIVIRYGLVTNEIAMVQARGRARADESTYVLVAHSGSGVIEHETVNDFREKMMYKAIHCVQNMKPEEYAHKILELQMQSIMEKKMKTKRNIAKHYKNNPSLITFLCKNCSVLACSGEDIHVIEKMHHVNMTPEFKELYIVRENKALQKKCADYQINGEIICKCGQAWGTMMVHKGLDLPCLKIRNFVVVFKNNSTKKQYKKWVELPITFPNLDYSECCLFSDED

SEQ ID NO: 3 - DNA, 3434 bp, Homo sapiens
gccccgctgcccacctgcccgcctgcccacctgcccaggtgcgagtgcagccccgcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgcaaaaaaaaaaaaaa

SEQ ID NO: 4 - protein, 1025 aa, Homo sapiens
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIENLSQVDGPQVEEQLLSTTVQPNLEKEVWGMENNSSESSFADSSVVSESDTSLAEGSVSCLDESLGHNSNMGSDSGTMGSDSDEENVAARASPEPELQLRPYQMEVAQPALEGKNIIICLPTGSGKTRVAVYIAKDHLDKKKKASEPGKVIVLVNKVLLVEQLFRKEFQPFLKKWYRVIGLSGDTQLKISFPEVVKSCDIIISTAQILENSLLNLENGEDAGVQLSDFSLIIIDECHHTNKEAVYNNIMRHYLMQKLKNNRLKKENKPVIPLPQILGLTASPGVGGATKQAKAEEHILKLCANLDAFTIKTVKENLDQLKNQIQEPCKKFAIADATREDPFKEKLLEIMTRIQTYCQMSPMSDFGTQPYEQWAIQMEKKAAKEGNRKERVCAEHLRKYNEALQINDTIRMIDAYTHLETFYNEEKDKKFAVIEDDSDEGGDDEYCDGDEDEDDLKKPLKLDETDRFLMTLFFENNKMLKRLAENPEYENEKLTKLRNTIMEQYTRTEESARGIIFTKTRQSAYALSQWITENEKFAEVGVKAHHLIGAGHSSEFKPMTQNEQKEVISKFRTGKINLLIATTVAEEGLDIKECNIVIRYGLVTNEIAMVQARGRARADESTYVLVAHSGSGVIEHETVNDFREKMMYKAIHCVQNMKPEEYAHKILELQMQSIMEKKMKTKRNIAKHYKNNPSLITFLCKNCSVLACSGEDIHVIEKMHHVNMTPEFKELYIVRENKALQKKCADYQINGEIICKCGQAWGTMMVHKGLDLPCLKIRNFVVVFKNNSTKKQYKKWVELPITFPNLDYSECCLFSDED

(19) United States
(12) Patent Application Publication
(10) Pub. No.: US 2011/0165600 A1
(43) Pub. Date: Jul. 7, 2011

(54) DIAGNOSIS METHOD AND DIAGNOSIS KIT FOR DERMATOMYOSITIS

(76) Inventors: Masataka Kuwana, Tokyo (JP); Shinji Sato, Tokyo (JP); Takashi Fujita, Kyoto (JP)

(21) Appl. No.: 13/059,444

(22) PCT Filed: Jul. 31, 2009
(86) PCT No.: PCT/JP2009/063633

§ 371 Date: Feb. 17, 2011

(30) Foreign Application Priority Data
Sep. 1, 2008 (JP) 2008-223789

(51) Int. Cl. G01N 33/53; C12N 9/00

(52) U.S. Cl. 435/7.92; 435/183

(57) ABSTRACT

An object of the present invention is to identify an antigen corresponding to anti-CADM-140 antibody, produce a recombinant protein, and establish an assay system by ELISA or the like. The present invention provides a kit for diagnosing dermatomyositis containing an MDA5 protein shown in SEQ ID NO: 4 or a fragment thereof that is recognized by anti-CADM-140 antibody.

### TECHNICAL FIELD

The present invention relates to a method and a kit for diagnosing dermatomyositis, and use of an antigenic protein for diagnosing dermatomyositis.

### BACKGROUND ART

A basic characteristic of collagen disease that causes it to be regarded as an autoimmune disease is the production of autoantibodies against various cellular components. It is known that many of the antigens corresponding to such autoantibodies are enzymes and regulatory factors that are essential to life processes. Investigating the molecular structures of the autoantibodies and antigens corresponding thereto (autoantigens), and their biological functions is expected to contribute to the elucidation of the pathogenesis of connective tissue diseases.

Polymyositis/dermatomyositis (PM/DM) is an inflammatory myopathy in which proximal muscle weakness and muscular pain caused by skeletal muscle inflammation predominate. Particularly, when typical skin symptoms such as heliotrope rash and Gottron's papules are observed, the patient is diagnosed with DM. Polymyositis/dermatomyositis is one of the autoimmune diseases, and various autoantibodies are known to develop. The presence of antibodies in the sera of PM/DM patients has been reported; such antibodies include anti-aminoacyl tRNA synthetase antibody (anti-ARS antibody), anti-SRP antibody, anti-Mi-2 antibody, and like antibodies specifically detected in myositis patients, as well as anti-U1RNP antibody, anti-SS-A antibody, and like autoantibodies associated with myositis. It is known that patients who are positive for the same myositis-specific autoantibody exhibit similar clinical characteristics; this is clinically useful for diagnosis, classification of disease types, selection of appropriate treatment methods, and estimation of prognosis.

In contrast, autoantibody negativity was considered to be one of the characteristics of clinically amyopathic DM (C-ADM), which is a sub-type of PM/DM. The presence of autoantibodies specific to C-ADM was unknown. It was known that C-ADM is resistant to clinical treatment and associated with poor prognostic rapidly progressive interstitial lung disease (RP-ILD). To save the life of C-ADM patients with RP-ILD, the efficacy of potent treatment from an early stage using a combination of high dose steroid therapy and immunosuppressive drug administration has been reported and recommended. For this reason, early diagnosis of C-ADM with associated RP-ILD is clinically important, and the establishment of new indicators that are useful for early diagnosis has been desired.

In light of the above background, the present inventors investigated the sera of normal healthy controls, those of patients with connective tissue diseases including C-ADM, and those of patients with idiopathic pulmonary fibrosis, according to an immunoprecipitation (IPP) method. The inventors found that a new autoantibody capable of recognizing a 140-kDa protein is present in the sera of C-ADM patients, and termed the new autoantibody “anti-CADM-140 antibody” (Non-Patent Literature 1 (NPL 1)).

The anti-CADM-140 antibody was not detected in any of the patients with connective tissue diseases other than C-ADM, patients with idiopathic pulmonary fibrosis (IPF), and normal healthy controls. Clinically, significantly frequent development of RP-ILD in anti-CADM-140 antibody-positive patients was observed, which suggests a correlation between the anti-CADM-140 antibody and RP-ILD (Non-Patent Document 2).

This phenomenon is considered to be useful for early diagnosis and treatment selection for extremely poor prognostic C-ADM with associated RP-ILD, and is thus expected to improve its prognosis.

MDA5 has a SNP (http://www.ncbi.nlm.nih.gov/SNP/snp_ref.cgi?locusId=64135). The amino acid sequence and the base sequence thereof are available under NCBI Accession Number NM_{—}022168. Patent Literature 1 (PTL 1) discloses the relationship between MDA5 and cancer.

The present inventors published Non-Patent Literature 3 (NPL 3), which discloses a technique to which the present invention pertains.

### CITATION LIST

Patent Literature

PTL 1: Japanese Unexamined Patent Document No. 2003-531581

Non-Patent Literature

NPL 1: Arthritis Rheum. 46(9): S398, 2003

NPL 2: Arthritis Rheum. 52(5): 1571-1576, 2005

NPL 3: Arthritis Rheum. 60(7): 2193-2200, 2009

### SUMMARY OF INVENTION

### Technical Problem

Anti-CADM-140 antibody has been generally measured according to an IPP method using S³⁵-labeled leukemia-derived K562 cell extracts or HeLa cells. Although this is a reliable measurement method with high sensitivity and high specificity, the method utilizes isotopes and requires complicated procedures. Accordingly, the measurement method has been used only in a limited number of laboratories.

To apply anti-CADM-140 antibody measurement to actual clinical diagnosis, it is necessary to establish a measurement system that enables easy measurement of large quantities of samples. Identification of an antigen corresponding to anti-CADM-140 antibody, preparation of a recombinant protein, and establishment of a measurement system by ELISA etc., are important to establish such a system.

### Solution to Problem

The present inventors used a HeLa cell cDNA library to clone an antigen gene corresponding to anti-CADM-140 antibody, and investigated the molecular sequence of the corresponding antigen protein. As a result, the inventors found that MDA5 (Melanoma Differentiation Associated Gene 5) is an antigen corresponding to anti-CADM-140 antibody and have accomplished the present invention based on this finding.

The present invention provides kits and methods for diagnosing dermatomyositis as itemized below.

Item 1. A kit for diagnosing dermatomyositis, comprising an MDA5 protein shown in SEQ ID NO: 4, or a fragment thereof that is recognized by anti-CADM-140 antibody.
Item 2. The kit for diagnosing dermatomyositis according to Item 1, comprising a C-terminal fragment of the MDA5 protein shown in SEQ ID NO: 2, or a fragment thereof that is recognized by anti-CADM-140 antibody.
Item 3. The kit according to Item 1, wherein the dermatomyositis is a clinically amyopathic dermatomyositis (C-ADM) associated with a high risk for developing rapidly progressive interstitial lung disease.
Item 4. The kit according to Item 1 used for measurement by enzyme-linked immunosorbent assay (ELISA).

Item 5. The kit according to Item 1, comprising:

(i) an antigen comprising an MDA5 protein or an immunogenic peptide thereof;
(ii) a medium suitable for use in a reaction between a sample of a subject and the antigen (i);
(iii) a reagent for detecting a complex of the antigen (i) and anti-CADM-140 antibody; and optionally
(iv) a reference sample not containing anti-CADM-140 antibody.
Item 6. A method for diagnosing dermatomyositis, comprising allowing a sample of a subject to react with an MDA5 protein or a fragment thereof that is recognized by anti-CADM-140 antibody; and diagnosing the subject as having dermatomyositis when the anti-CADM-140 antibody is detected in the sample.
Item 7. The method according to Item 5 wherein the sample is a blood, serum, or plasma sample.
Item 8. The method according to Item 6 wherein the anti-CADM-140 antibody in the sample is detected by ELISA.
Item 9. The method according to Item 6 comprising the following steps:
(i) depositing a specific amount of a peptide composition of the present invention on a plurality of wells of a microtiter plate;
(ii) diluting a sample of a subject suspected of having dermatomyositis, and adding the diluted sample to the wells;
(iii) washing the microtiter plate after incubation;
(iv) introducing a labeled anti-human immunoglobulin antibody to the wells of the microtiter plate; and
(v) detecting the amount of label bound to the antibody by comparison with a control.
Item 10. Use of an MDA5 protein or a fragment thereof that is recognized by anti-CADM-140 antibody to diagnose dermatomyositis.

### ADVANTAGEOUS EFFECTS OF INVENTION

According to the present invention, extremely poor prognostic clinically amyopathic dermatomyositis associated with a high risk for developing extremely poor prognostic rapidly progressive interstitial lung disease can be detected with high accuracy.

The present invention enables quick measurement of anti-CADM-140 antibody in many samples. This is useful for early diagnosis and treatment selection for C-ADM with associated RP-ILD, and is expected to improve the prognosis of this disease whose treatment method has not been established and which has been considered as an extremely poor prognostic disease. Furthermore, the accumulation of anti-CADM-140 antibody-positive samples will enable the analysis of foreign antigens, such as a virus that is the target of a specific autoantibody, and contribute to the elucidation of the pathogenesis of C-ADM with associated RP-ILD.

### BRIEF DESCRIPTION OF DRAWINGS

FIG. 1 shows the results of investigation of a reaction between a protein of Clone #8 (MDA5) as an antigen and the sera of C-ADM patients according to an immunoprecipitation method, which was performed after purifying the protein of Clone #8 (MDA5) and confirming the expression of the protein using an in vitro transcription/translation system. The Clone #8 (MDA5) reacted with all of the anti-CADM-140 antibody-positive serum samples, but it did not react with the sera of normal healthy controls.

FIG. 2 shows the results of investigation of a reaction between a recombinant protein of Clone #8 (MDA5) as an antigen, and the sera of anti-CADM-140 antibody-positive C-ADM patients, those of patients with other connective tissue diseases, and those of normal healthy controls, according to an immunoprecipitation method, which was performed after purifying the protein of Clone #8 (MDA5) and confirming expression of the protein using an in vitro transcription/translation system. The recombinant protein did not react with the sera of normal healthy controls or the sera of patients other than anti-CADM-140 antibody-positive C-ADM patients.

FIG. 3 shows the IPP-IB results. When anti-CADM-140 antibody was reacted with an MDA5-expressing African green monkey renal cell-derived COS7 cell extract, the resulting 140-kDa immunoprecipitate reacted with goat anti-MDA5 antibody.

FIG. 4 shows the immunoblotting results of the sera of anti-CADM-140 antibody-positive patients and those of normal healthy controls, obtained by using recombinant RIG-I, recombinant MDA5, and recombinant LGP-2 as antigens. The sera of anti-CADM-140 antibody-positive patients reacted only with the recombinant MDA5, whereas the sera of normal healthy controls (NHC) did not react at all.

FIG. 5 shows the ELISA results of the sera of anti-CADM-140 antibody-positive C-ADM patients, those of anti-CADM-140 antibody-negative C-ADM patients, those of PM/DM patients, those of scleroderma (SSc) patients, those of systemic lupus erythematosus (SLE) patients, those of ILD patients, and those of normal healthy controls, obtained by using MDA5 as an antigen substrate. The cutoff value is indicated by a horizontal line. The analytical sensitivity and analytical specificity of this ELISA system were 85% and 100%, respectively.

### DESCRIPTION OF EMBODIMENTS

A feature of the present invention is an antigen (MDA5) specifically recognized by anti-CADM-140 antibody, which was found by the present inventor. MDA5 is one type of RIG-I family protein. RIG-I family proteins participate in a specific immune response that includes interferon type I production. RIG-I family proteins are highly homologous and contain a DexD/H-box helicase domain. In addition to MDA5, RIG-I and LPG-2 are also known as RIG-I family proteins. These proteins are very similar in sequence and structure, and are thus likely to be cross-reactive. Accordingly, the reactivity between anti-CADM-140 antibody and RIG-I family proteins was also investigated. The binding of anti-CADM-140 antibody to MDA5 depends on the conformation of the antigen, and anti-CADM-140 antibody preferentially reacts with an MDA5 partially modified from its native structure. These factors made it difficult to determine that anti-CADM-140 antibody is an antibody to MDA5.

To identify anti-CADM-140 antibody, the present inventors purified an antigen protein corresponding thereto and tried to identify the antigen protein by mass spectrometry. However, it was impossible to purify a sufficient amount of the antigen protein, and identification was difficult. This was considered to be attributable to low amounts of antigen in the serum or to the affinity of the antigen-antibody reaction.

Further, in the early stages of C-ADM, it is difficult to distinguish the disease from other types of dermatomyositis, because C-ADM is difficult to diagnose and requires follow-up to carefully determine whether muscular symptoms are present or not.

However, the present inventors identified MDA5 specifically recognized by anti-CADM-140 antibody. As a result, it became clear that the clinical sensitivity and clinical specificity of the ELISA assay of dermatomyositis (C-ADM) are 69% and 99.6%, respectively. Although anti-CADM-140 antibody-negative C-ADM patients were previously difficult to diagnose or the diagnosis often took much time, the present invention enables quick diagnosis.

In this specification, “dermatomyositis” includes what is referred to as DM and clinically amyopathic dermatomyositis (C-ADM), which is a subtype of DM, but does not include polymyositis PM. Thus, the present invention provides a kit and a method for diagnosing whether the subject has DM or C-ADM or other diseases. DM is an inflammatory myopathy in which characteristic skin symptoms such as heliotrope rash and Gottron's papules, and proximal muscle weakness and/or muscular pain caused by skeletal muscle inflammation predominate. DM is generally diagnosed according to the diagnostic criteria of Bohan & Peter (Bohan A, Peter J B. Polymyositis and dermatomyositis. N Engl J Med 1975; 292: 344). However, C-ADM, which refers to the case in which cutaneous lesions characteristic of DM are exhibited but muscle weakness is not exhibited, does not meet the criteria of Bohan & Peter, and has been undiagnosable. In 2002, Sontheimer proposed new classification criteria in which a case of typical skin lesions without muscle weakness is called “C-ADM” (clinically amyopathic dermatomyositis), and “C-ADM” is classified as a subtype of DM [Sontheimer R D: Would a new name hasten the acceptance of amyopathic dermatomyositis (dermatomyositis sine myositis) as a distinctive subset within the idiopathic inflammatory dermatomyopathies spectrum of clinical illness? J Am Acad Dermatol. 2002; 46: 626-36]. The target DM to be diagnosed according to the present invention includes those that meet the diagnostic criteria of Bohan & Peter or meet the classification criteria of Sontheimer.

The diagnostic kit and the diagnostic method of the present invention use an MDA5 protein or a fragment thereof recognized by anti-CADM-140 antibody to detect anti-CADM-140 antibody (hereinafter the “fragment recognized by anti-CADM-140 antibody” is sometimes simply referred to as an “immunogenic peptide”). Since the C-terminal 523-amino-acid sequence fragment (SEQ ID NO: 2) of MDA5 protein shown in SEQ ID NO: 4 binds to anti-CADM-140 antibody, an epitope recognized by anti-CADM-140 antibody is present in the C-terminal region of MDA5 protein. Insofar as this epitope is maintained, the MDA5 protein or the immunogenic peptide thereof may be a modified MDA5 protein or immunogenic peptide thereof that has one or more amino acid deletions, substitutions, additions, or insertions in a non-epitopic region thereof. The epitope typically consists of about 5 to about 10 amino acids, and particularly about 5 to about 8 amino acids. For example, the epitope of MDA5 can be determined by synthesizing a specific number of peptides (for example, 10 peptides at a time) while shifting by a fixed number of amino acids (for example, 5 amino acids) from the N-terminus of the C-terminal fragment of MDA5 shown in SEQ ID NO: 2 (for example, prepare a peptide consisting of the first to tenth amino acids, a peptide consisting of the sixth to 15th amino acids, a peptide consisting of the 11th to 20th amino acids, a peptide consisting of the 16th to 25th amino acids, . . . ), and then investigating the reactivity of each of the peptide fragments with anti-CADM-140 antibody. When the epitope is determined or the location of the epitope is narrowed down, the MDA5 protein (immunogenic peptide) fragment recognized by anti-CADM-140 antibody can be shortened. A shorter immunogenic peptide is preferable from the viewpoint of ease of production. A peptide consisting of 40 or less amino acids, 30 or less amino acids, or 20 or less amino acids, particularly about 5 to about 10 amino acids, is preferable. RNA helicase is encoded by MDA5.

MDA5 is known to have an SNP. The MDA5 protein or the immunogenic peptide that is used in the present invention may have any type of SNP. SEQ ID NO: 3 represents a base sequence of MDA5. SEQ ID NO: 1 represents a base sequence encoding a C-terminal fragment of the MDA5 fragment shown in SEQ ID NO: 2. However, the MDA5 of other amino acid sequences/base sequences that are related by SNP, or alleles of MDA5 are also included in the scope of “MDA5” in this specification.

The MDA5 or the immunogenic peptide of the present invention can be expressed as a recombinant protein and produced by genetic engineering, or can be produced by chemical synthesis. Particularly, when the immunogenic peptide has a short sequence consisting of preferably 50 or less amino acids, more preferably 30 or less, even more preferably 20 or less, and particularly, 5 to 10 amino acids, chemical synthesis (a solid or liquid phase synthesis) is preferably used to produce the peptide. The MDA5 or the immunogenic peptide thereof may be used in the form of a monomer, and may be crosslinked by a polyfunctional crosslinking agent such as glutaraldehyde, or can be produced by genetic engineering by using DNA encoding a peptide in which immunogenic peptides are connected in tandem.

The anti-CADM-140 antibody may be obtained from any sample such as the blood, serum, plasma, cerebrospinal fluid, and lymph of a subject. Blood, serum, and plasma are preferable, and serum is particularly preferable as the sample.

The kit for diagnosing dermatomyositis of the present invention contains an MDA5 protein or an immunogenic peptide thereof. The kit may contain a substance for detecting an antigen-antibody complex of an MDA5 protein or an immunogenic peptide thereof, and anti-CADM-140 antibody. Examples of such substances include anti-human immunoglobulin antibodies that are labeled with peroxidases such as horseradish peroxidase (HRP), enzyme labels such as β-galactosidase, alkaline phosphatase, and luciferase, fluorescent labels such as FITC (fluorescein isothiocyanate) and RITC (tetramethylrhodamin isothiocyanate), or any other suitable labels, and particularly anti-human IgG antibodies labeled therewith.

Immunoassay is preferably used for the diagnostic kit and the diagnostic method of the present invention. Examples of the immunoassay include enzyme immunoassay (EIA), ELISA, fluoroimmunoassay (FIA), chemiluminescent immunoassay, immunoblotting (IB), Western blotting, immunostaining, and like methods.

The MDA5 protein or the immunogenic peptide used in the present invention is preferably bonded to a solid phase. Examples of such solid phases include agarose, wells of a microtiter plate, latex particles, and the like. Specific examples of the ELISA method include competitive immunoassay, sandwich immunoassay, and the like.

According to the method for diagnosing dermatomyositis of the present invention, a sample obtained from a subject is brought into contact with an MDA5 protein or an immunogenic peptide thereof to detect the presence or absence of an antigen-antibody complex of MDA5 or an immunogenic peptide thereof and anti-CADM-140 antibody by a physical or chemical method.

The method for diagnosing dermatomyositis of the present invention comprises, for example, the following steps:

(i) depositing a specific amount of the peptide composition of the present invention on a plurality of wells of a microtiter plate;
(ii) diluting a blood sample (serum or plasma) of a subject suspected of having dermatomyositis, and adding the diluted sample to the wells;
(iii) washing the microtiter plate after incubation;
(iv) adding human immunoglobulin (e.g., IgG) labeled with an enzyme label, a fluorescent label, or the like, to the wells of the microtiter plate; and
(v) detecting the amount of label bound to human immunoglobulin (the amount of substrate acted upon by an enzyme in the case of human immunoglobulin labeled with an enzyme label), compared to a control.

In a preferred embodiment, the diagnostic kit of the present invention may contain:

(i) an antigen comprising an MDA5 protein or an immunogenic peptide thereof (the antigen may be adhered to wells of a microtiter plate etc., and may further be blocked with a blocking agent such as skim milk, or an albumin such as bovine serum albumin (BSA) or I);
(ii) a medium suitable for the reaction between a sample of a subject and the antigen (i) (for example, buffers such as PBS);
(iii) a reagent for detecting a complex of the antigen (i) and anti-CADM-140 antibody (for example, a labeled anti-human immunoglobulin antibody bound to anti-CADM-140 antibody); and optionally
(iv) a reference sample not containing anti-CADM-140 antibody.

The “reference sample not containing anti-CADM-140 antibody” includes, for example, blood, serum, and plasma samples of normal healthy controls.

When the anti-human immunoglobulin antibody is labeled with peroxidase, anti-CADM-140 antibody can be detected by a process comprising adding a color development substrate such as tetramethylbenzidine; stopping the reaction with H₂SO₄; and measuring absorbance (450 nm: OD450) using a plate reader.

By testing many samples using the diagnostic kit of the present invention and comparing the obtained results with other clinical observations, more accurate criteria for determining whether a subject has DM, based on the measured amount of anti-CADM-140 antibody can be established.

### EXAMPLES

The present invention is described below in more detail.

### Example 1

### Method for Early Diagnosis of C-ADM

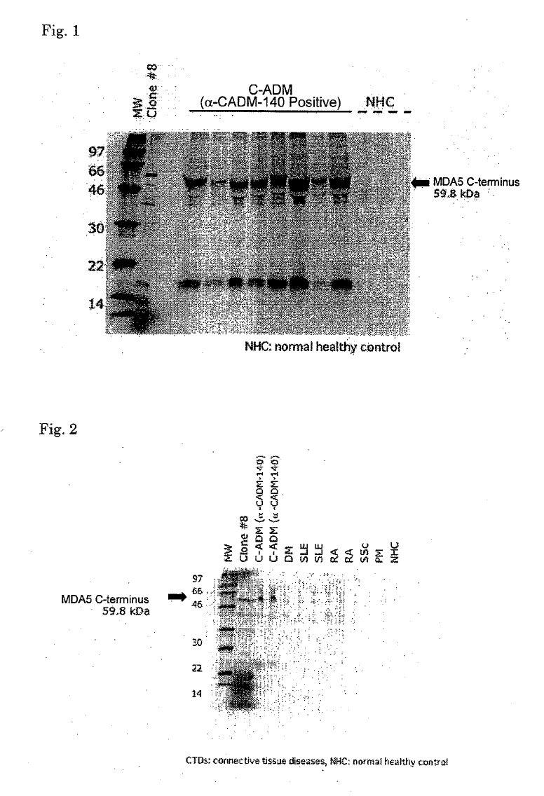

An antigenic protein was expressed in E. coli by using an E. coli (XL1-Blue MRF) expressing lambda ZAP phage prepared by using a HeLa cell cDNA library. The expressed protein was transferred to a nitrocellulose membrane, and reacted with anti-CADM-140 antibody-positive serum to isolate positive clones. Next, the reactivity of the obtained positive clones with 10 serum samples of anti-CADM-140 antibody-positive C-ADM patients, 14 serum samples of anti-CADM-140 antibody-negative persons (two C-ADM samples, two PM samples, one sample each of systemic lupus erythematosus, scleroderma, and interstitial lung disease, and seven samples of normal healthy controls) was investigated. Among the 9 clones that were obtained, Clone #8 reacted with anti-CADM-140 antibody-positive serum samples at high frequency, i.e. 9 out of 10 anti-CADM-140 antibody-positive C-ADM serum samples. After the protein was purified and expression was confirmed using an in vitro transcription/translation assay, the reactivity of the sera of C-ADM patients with the protein as an antigen was investigated by immunoprecipitation. Clone #8 reacted with all of the anti-CADM-140 antibody-positive serum samples, but did not react with the sera of normal healthy controls (FIG. 1). Further, Clone #8 did not react with the sera of patients with connective tissue diseases other than anti-CADM-140 antibody-positive C-ADM (FIG. 2). Further, to determine the base sequence of Clone #8, plasmid DNA was excised from phage DNA from which the clone was obtained. The plasmid DNA was purified to determine the base sequence. Finally, a homology search was performed for the obtained base sequence, and the sequence was perfectly matched to the C-terminal sequence of MDA5.

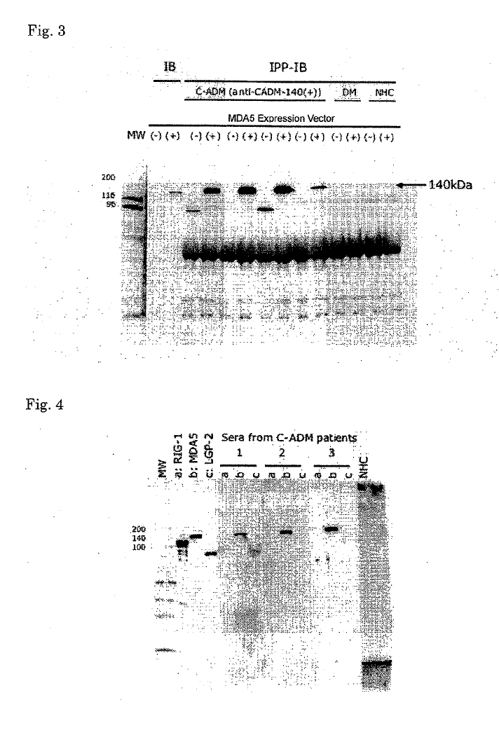

An anti-CADM-140 antibody-positive serum was reacted with an MDA5-expressing African green monkey renal cell-derived COST cell extract by the IPP-IB method. The reactivity of 140 kDa of the obtained immunoprecipitate with goat anti-MDA5 antibody was investigated. When MDA5 was expressed, the immunoprecipitate reacted with goat anti-MDA5 antibody (FIG. 3). Further, the reactivity of anti-CADM-140 antibody-positive sera and the sera of normal healthy controls with recombinant purified proteins of RIG-I family RIG-I, MD5, and LGP-2 as antigens was investigated. The sera of anti-CADM-140 antibody-positive patients reacted only with recombinant MDA5, whereas the sera of normal healthy controls did not react with recombinant MDA5 (FIG. 4). It became clear from the above results that the antigen corresponding to anti-CADM-140 antibody is MDA5.

Further, the present inventors coated a 96-well plate with purified recombinant MDA5 as an antigen to establish an ELISA for an anti-ACDM-140 antibody assay. The diagnostic sensitivity and diagnostic specificity of the anti-CADM-140 antibody assay method were investigated.

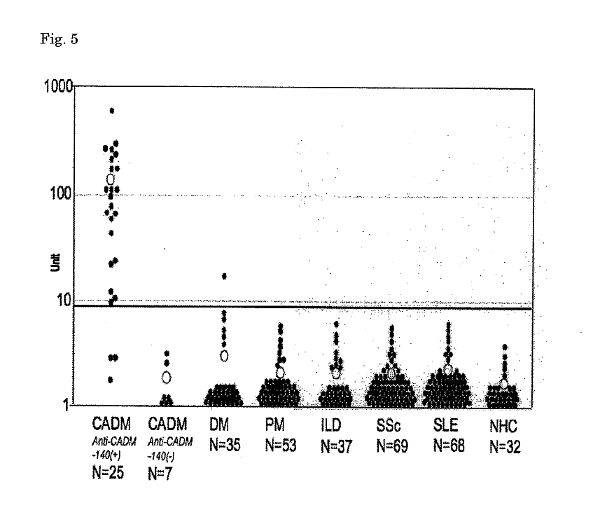

More specifically, recombinant MDA5 was immobilized on a 96-well ELISA plate in an amount of 0.05 μg/well and blocked with 3% BSA. Serum samples (each 250-fold diluted) were dispensed to the plate in an amount of 100 μl per well, and allowed to react at room temperature for 2 hours. As a secondary antibody, 5000-fold diluted peroxidase conjugated goat anti-human-IgG was used. Tetramethylbenzidine (1 mg/ml) was added as a color development substrate, and the reaction was stopped with H₂SO₄. The absorbance was measured with a plate reader (450 nm: OD₄₅₀). Using this assay system, the serum samples of C-ADM-containing collagen disease patients, those of interstitial lung disease (ILD) patients, and those of normal healthy controls were measured. The results showed that the reactivity of the sera of the anti-CADM-140 antibody-positive PM/DM patients was higher than that of the anti-CADM-140 antibody-negative PM/DM patients, scleroderma (SSc) patients, systemic lupus erythematosus (SLE) patients, ILD patients, and normal healthy controls (FIG. 5). When the average+up to 10 times the standard derivation of normal healthy controls was defined as the normal cutoff range, the analytical sensitivity and the analytical specificity measured by this ELISA assay system were 85% and 100%, respectively. Thus, the results clearly show that this assay is an anti-CADM-140 antibody detection method with excellent sensitivity and specificity.

1. A kit for diagnosing dermatomyositis, comprising an MDA5 protein shown in SEQ ID NO: 4, or a fragment thereof that is recognized by anti-CADM-140 antibody.

2. The kit for diagnosing dermatomyositis according to claim 1, comprising a C-terminal fragment of the MDA5 protein shown in SEQ ID NO: 2, or a fragment thereof that is recognized by anti-CADM-140 antibody.

3. The kit according to claim 1, wherein the dermatomyositis is a clinically amyopathic dermatomyositis (C-ADM) associated with a high risk for developing rapidly progressive interstitial lung disease.

4. The kit according to claim 1 used for measurement by enzyme-linked immunosorbent assay (ELISA).

5. The kit according to claim 1, comprising:
(i) an antigen comprising an MDA5 protein or an immunogenic peptide thereof;
(ii) a medium suitable for use in a reaction between a sample of a subject and the antigen (i);
(iii) a reagent for detecting a complex of the antigen (i) and anti-CADM-140 antibody and; and optionally
(iv) a reference sample not containing anti-CADM-140 antibody.

6. A method for diagnosing dermatomyositis, comprising allowing a sample of a subject to react with an MDA5 protein or a fragment thereof that is recognized by anti-CADM-140 antibody; and diagnosing the subject as having dermatomyositis when the anti-CADM-140 antibody is detected in the sample.

7. The method according to claim 6 wherein the sample is a blood, serum, or plasma sample.

8. The method according to claim 6 wherein the anti-CADM-140 antibody in the sample is detected by ELISA.

9. The method according to claim 6 comprising the following steps:
(i) depositing a specific amount of a peptide composition of the present invention on a plurality of wells of a microtiter plate;
(ii) diluting a sample of a subject suspected of having dermatomyositis, and adding the diluted sample to the wells;
(iii) washing the microtiter plate after incubation;
(iv) introducing a labeled anti-human immunoglobulin antibody to the wells of the microtiter plate; and
(v) detecting the amount of label bound to the antibody by comparison with a control.

10. Use of an MDA5 protein or a fragment thereof that is recognized by anti-CADM-140 antibody to diagnose dermatomyositis.
